(19) United States
(12) Patent Application Publication
(10) Pub. No.: US 2024/0115762 A1
(43) Pub. Date: Apr. 11, 2024

(54) MAGNETICALLY ALIGNED POLYMERIC MICROFIBERS

(71) Applicant: Worcester Polytechnic Institute, Worcester, MA (US)

(72) Inventors: Jeannine Coburn, Boylston, MA (US); Melissa Wojnowski, Mendon, MA (US)

(21) Appl. No.: 18/377,791

(22) Filed: Oct. 7, 2023

Related U.S. Application Data
(60) Provisional application No. 63/414,051, filed on Oct. 7, 2022.

(51) Int. Cl. A61L 27/22; A61L 27/04; A61L 27/52

(52) U.S. Cl. CPC A61L 27/227; A61L 27/042; A61L 27/52; A61L 2400/06; A61L 2430/32

(57) ABSTRACT

Magneto-responsive properties are traditionally imparted to scaffold systems via integration of iron oxide-based magnetic nanoparticles (MNPs), yet poor understanding of long-term MNP toxicity presents a significant translational challenge. Given the demonstrated iron-binding capacity of silk fibroin (SF), passive chelation of ferric iron ions is explored herein as an alternative, MNP-free approach for magnetic functionalization of silk fibroin (SF)-based biomaterials. SF microfibers treated with aqueous ferric chloride (FeCl₃) exhibit significantly increased iron content relative to the nascent protein.

### RELATED APPLICATIONS

This patent application claims the benefit under 35 U.S.C. § 119(e) of U.S. Provisional Patent App. No. 63/414,051 filed Oct. 7, 2022, entitled “MAGNETICALLY ALIGNED POLYMERIC MICROFIBERS,” incorporated herein by reference in entirety.

### BACKGROUND

An alarming disparity persists between the incidence and prevalence of traumatic spinal cord injury (SCI). With minimal spontaneous neuroregeneration post-injury and no well-established treatment or cure, an annual incidence of 17,000 new cases/year has left as many as 400,000 patients living with an unresolved chronic SCI in the United States alone. Per the mantra “time is spine,” the standard of care for SCI emphasizes early intervention to mitigate the rapid progression of secondary damage and stabilize the injury site (e.g., surgical decompression, supplemental pharmacological interventions). Unfortunately, the neurorecovery achieved after injury stabilization is primarily limited to the spared nerve tissue. Among patients that survive the initial trauma of the primary injury, incomplete tetraplegia is the most frequent neurological outcome at the time of hospital discharge (47.2% of patients), followed by incomplete/complete paraplegia and complete tetraplegia. Less than 1% of SCI patients will completely recover neurological functions lost during their injury, meaning the prognosis for nearly all SCI patients is permanent, irrecoverable paralysis.

### SUMMARY

An injectable silk fibroin microfibers (mSF) scaffold with magneto response properties is responsive to magnetic fields for facilitating alignment and subsequent growth when implanted in a therapeutic treatment site. A gel or polymer based medium imparts a fluidic injectability for patient delivery, and subsequently binds the microfibers in an aligned orientation following the magnetic response. Ferric iron (Fe³⁺) chelation or introduction from an aqueous solution imparts the magnetic properties without the potential toxicity of ferrous iron (Fe²⁺). Magnetic response is achieved with modest iron presence, and the aqueous iron washes excess away cleanly.

Configurations herein are based, in part, on the observation that human nerve tissue, and in particular spinal cord injury (SCI) is challenging to heal and regenerate. Unfortunately, conventional approaches to nerve tissue treatment suffer from the shortcoming that it is difficult to implant or place a scaffold or cell regrowth medium in an injury site in a manner conducive to cell regeneration. Accordingly, configurations herein substantially overcome the shortcomings of conventional nerve cell/CNS (Central Nervous System) regeneration by providing a scaffold medium such as silk fibroin microfibers (mSF) and binding with iron for imparting a magnetic response for facilitating subsequent fibrous alignment in an injury site. An aqueous gel medium transports the iron-bond mSF via injection to a spinal cord or nerve injury, and alignment is achieved through application of a modest magnetic field at the treatment site, following which the gel medium maintains the mSF in alignment conducive for healing and regrowth.

In further detail, an example configuration of forming an injectable medical scaffolding includes hydrolyzing silk fibroin in a sodium hydroxide solution to fabricate silk fibroin microfibers (mSF), neutralizing the mSF with an acidic wash, and rinsing and lyophilizing the mSF to form a powder. Transport, storage and deployment may occur, and upon a therapeutic need, resuspending the mSF in a ferric chloride solution for 12-48 hours followed by water washing to form a magneto responsive mSF. Combining the magneto responsive mSF with a hydrophilic crosslinking gel for maintaining magnetic alignment, and administering the gel based, iron bound mSF to an injury site, followed by introduction of a magnetic field for alignment serves as a foundation for SCI repair.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a process flow of development of the iron bound mSF for use in SCI repair;

FIG. 2 is a graph of iron-bound mSF and untreated mSF showing magneto responsiveness;

FIGS. 3A and 3B show images of non-iron bound mSF and magnetically aligned mSF; and

FIG. 4 shows a graph of magnetic response of Fe³⁺-mSF when exposed to external magnetic fields.

### DETAILED DESCRIPTION

The magneto-responsive microfibers disclosed herein can serve as an injectable neuroregenerative scaffold that can be aligned in situ, post-injection, thereby providing a support structure to guide injury-bridging nerve growth in spinal cord injuries (SCI) without requiring invasive, traumatic implantation procedures. Such a material would ultimately increase the effectiveness of cell therapy in regenerating functional nerve tissue in SCI patients, facilitating recovery from this currently incurable injury.

In native tissue, the extracellular matrix (ECM) not only serves as a passive scaffolding system for resident cells, but furthermore functions as an active sensor, actuator, and regulator of cellular behaviors. By orchestrating the presentation of biomechanical, biochemical, and biomolecular signals within the extracellular microenvironment, the ECM modulates the growth, proliferation, and differentiation of resident cells for coordinated, functionally integrative tissue development. The ubiquity of this active interplay between cells and their environment suggests that dynamic scaffolding systems are critical to achieving regenerative outcomes in tissue-engineered (TE) constructs. To this end, stimuli-responsive biomaterials represent an exciting prospect in scaffold design. This subset of biomaterials is distinguished by their capacity to alter their physiochemical properties in response to exogenous variables or events, ultimately accommodating a more dynamically nuanced recapitulation of native tissues as compared to their static counterparts. While physiologically inherent factors have been explored as potential endogenous stimuli (e.g., pH, enzymes, small biological molecules, redox potentials), biomaterials that respond to externally generated, easily attenuated stimuli currently hold the greatest translational promise. When integrated into scaffolds, these stimuli-responsive biomaterials endow remotely actuated mechanisms for in situ modulation and adaptation of the local physiological microenvironment, facilitating non-invasive, user-guided tunability of pro-regenerative cues presented within the TE construct.

Magnetic stimulation is a particularly attractive example of one such exogenous mechanism: not only are magnetic fields easily generated and attenuated, but their resolution and precision are largely pre-served with tissue penetration, as well, lending well to clinical applications. Capable of inducing both pro-regenerative cellular behaviors and mechanical actuation of the surrounding scaffold, magnetic stimulation has demonstrated synergistic benefits in stimuli responsive platforms for vascular, cardiac, neural, bone, and musculoskeletal TE constructs.

With regard to scaffold design, magneto-responsive properties are typically achieved via functionalization of existing biomaterials with magnetic nanoparticles (MNPs). These MNPs are either blended (“doped”) into the respective polymers prior to scaffold fabrication, introduced as a coating post-fabrication via covalently bonding to the biomaterial surface, or precipitated out of aqueous metal ion solutions and deposited onto the scaffold during fabrication (“in situ precipitation”). For biomedical applications, MNPs are traditionally synthesized from iron-based oxides, particularly magnetite (Fe₃O₄) or its oxidized form, maghemite (γ-Fe₂O₃). Relative to other metal oxides, iron-based oxides exhibit particularly high magnetic saturation levels (i.e., high magnetic moment per unit volume) that impart significant magneto-responsiveness to functionalized scaffolds with limited increase in metal content. Furthermore, as nanoparticles, iron-oxides often demonstrate little or negligible magnetic remanence (i.e., induced magnetization that remains on removal of the external field), mitigating agglomeration-associated risks of inhibited functionality and off-target accumulation.

With regards to biocompatibility, physiological iron levels are regulated via highly adaptive homeostatic mechanisms that have evolved per the metal's unique duality as an essential micronutrient. Consequently, risks of metal-induced toxicity are more effectively alleviated for iron-based oxides than for other metal-based oxides. Metal toxicity and free radical production considered, ferric oxides are generally considered more biocompatible than ferrous oxides. However, both ferric and ferrous iron oxides have been shown to induce greater metal toxicity and oxidative stress than iron alone. Ongoing efforts to increase the biocompatibility of magneto-responsive scaffolds have explored modifications to MNP geometries and chemical compositions as well as alternatives to iron-oxide-based reagents, yet little research into iron-only functionalization schemes has been conducted in the development of magneto-responsive biomaterials.

When considered in the treatment of SCI, the development and clinical translation of a cure for SCI is largely hindered by the limited neuroregeneration and rapid neurodegeneration that occurs in the event of significant neuronal injury or death (i.e., traumatic SCI). Neurons are post-mitotic (i.e. will not divide to replace damaged or dying neurons), and their innate neuroregenerative capacity is easily repressed in inhibitory environments, including the injury site that is created by traumatic damage to spinal cord tissue. Accordingly, trends in clinical trials and preclinical research indicate intra-lesion, biomaterial-mediated cell therapy delivery as the most promising strategy for translationally efficacious neuroregeneration. While therapeutic cell transplantation replaces endogenous cell populations damaged or destroyed by injury, biomaterials regulate the biomechanical and biomolecular profile of the injury microenvironment, alleviating environmental inhibitors of innate neuroregeneration and promoting cell migration, maturation, and organization across the injury site. Unfortunately, both implantable and injectable biomaterial delivery vehicles innately limit the host-integrative neuroregenerative potential of cell therapies: the former disrupt the protective barrier function of the lesion-encompassing glial scar and exacerbate counter-productive neuroinflammation, while the latter lack aligned architecture to orienting lesion-bridging neurite outgrowth from transplanted cells, critical to directing their reconnection with spared nerve tissue in disrupted neural pathways. Thus, there is an unmet need for a biomaterial delivery vehicle that converts the cavitated SCI lesion into a pro-regenerative environment conducive to lesion-bridging, functionally integrative axonal regrowth without acute disruption of the glial scar.

A major advantage of the proposed magneto-responsive Fe³⁺-chelated mSF is its ability to serve as an injectable nerve guidance architecture, thereby decreasing counterproductive neurodegenerative inflammation (minimally invasive delivery mechanism) and increasing organized, functionally integrative neuroregeneration across the injury site (aligned scaffold architecture).

Configurations described below demonstrate passive chelation of ferric iron is investigated as an alternative to MNP-based functionalization methods for imparting magneto-responsive properties to silk fibroin (SF)-based biomaterials. Derived from Bombyx mori (B. mori) silkworm cocoons, SF is a biocompatible, biodegradable, FDA-approved natural biomaterial with an innately fibrous structure, lending well to applications as guidance architectures for directing hierarchically organized tissue regeneration within TE constructs. Furthermore, the significant reactive residue content of the SF protein affords an abundance of opportunity for chemical modification, including ligation sites for heavy metal chelation. Of note, SF has demonstrated a remarkable capacity for iron chelation, particularly in the ferric oxidation state, with tyrosine-contributed phenolic hydroxyl groups identified as likely coordination sites within nascent SF. However, the magneto-responsive properties of ferric iron-chelated SF remain uninvestigated, suggesting potential for, MNP-free mechanism for magnetic functionalization of mSF.

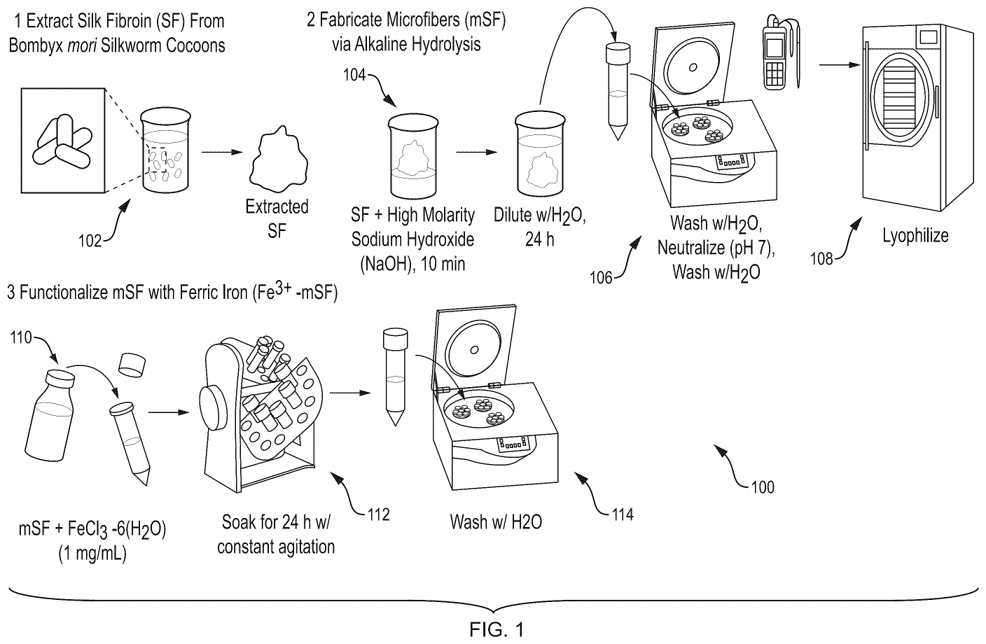

FIG. 1 is a process flow 100 of development of the iron bound mSF for use in SCI repair. The method for forming a scaffold amenable to nerve cell regrowth includes providing a scaffolding compound based on a potential for regrowth of nerve tissue, and adding an iron solution to the scaffolding compound for forming a magneto responsive scaffold adapted for implantation adjacent damaged nerve tissue.

Referring to FIG. 1, at step 102, silk fibroin microfibers (mSF) were fabricated via alkaline hydrolysis of sericin-extracted B. mori silk fibroin. Unlike regenerated SF fibers, which are fabricated by electrospinning, molding, or otherwise reforming solutions of dissolved SF into fibril geometries, SF fibers produced via controlled hydrolysis of nascent SF retain nanofibril structures inherent to the as-spun architecture.

At step 104, sodium hydroxide (NaOH) solution molarity and hydrolysis duration were manipulated to experimentally identify appropriate conditions for producing mSF of consistent length. Silk fibroin microfibers (mSF) were fabricated via alkaline hydrolysis of SF. Briefly, extracted SF was initially hydrolyzed in a 17.5 M sodium hydroxide (NaOH) solution (1.4% w/v SF) for 10 min, diluted to 6.25 M NaOH with ultrapure water (0.9% w/v SF), and allowed to react for an additional 24 h (ambient conditions).

The post-hydrolysis mSF product was washed with ultrapure water, adjusted to neutral pH with 1.5 M hydrochloric acid (HCl), washed again with ultrapure water, as depicted at step 106, and lyophilized, as shown at step 108. Following lyophilization, mSF were resuspended at 10% w/v in a 2 mM ferric chloride hexahydrate solution (FeCl₃·6(H₂O)), as disclosed at step 110 and reacted for 24 h with constant agitation (ambient conditions), as shown at step 112, after which the post-chelation Fe³⁺-mSF product was washed with ultrapure water (step 114). Magneto-responsive behavior of Fe³⁺-mSF was preliminarily characterized in a static magnetic field of clinically relevant strength (1.4 T, approximately equivalent to MRI field strength). Briefly, Fe³⁺-mSF were resuspended in an aqueous poly(ethylene glycol) diacrylate (PEG-DA) solution supplemented with photoinitiator Irgacure®2959, pipetted (100 μL) onto a glass cover slip positioned atop a diametric neodymium magnet (centered at the north/south polar interface), and allowed to align in the liquid phase, light protected, for 0.17, 0.33, 0.5, 1, 2, and 24 h prior to UV-initiated crosslinking of the PEG-DA solution. The gelation-preserved Fe³⁺-mSF orientation was imaged via ImageJ Directionality Analysis.

In practice, the disclosed approach provides an injectable scaffold system for spinal cord and nerve cell injury treatment using a silk fibroin microfiber (mSF) formed from a lyophilized powder and combined with an aqueous ferric chloride solution for imparting a magneto responsiveness to the mSF, formed with an injectable crosslinking hydrogel combined with the magneto responsive mSF for introduction into an injury site. The gel ideally has a solidification aspect such that once the mSF fibers are implanted, the gel permits movement responsive to the magnetic alignment, but is sufficiently persistent to maintain the alignment once the magnetic field is removed. Treatment is completed by a therapeutic magnetic source configured for applying between 100-400 mT for aligning the microfibers form a magnetic response, preferably from an adjacent epidermal location. The crosslinking hydrogel is adapted for maintaining the aligned orientation of the mSF following injection and alignment from the external magnetic source.

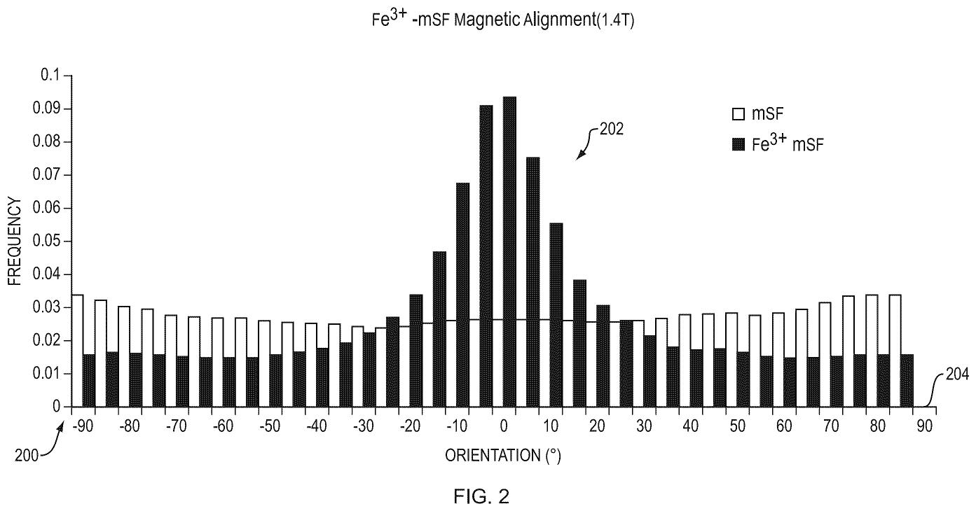

FIG. 2 shows a graph 200 of orientation on axis 204 of non-ferric iron chelated mSF orientation in absence of magnetic field vs ferric iron chelated mSF (‘Fe³⁺-mSF’) orientation in 1.4 T magnetic field. Uniformity of Fe³⁺-mSF alignment was quantified relative to the full width, half maximum (FWHM) of the ImageJ directionality analysis frequency histogram: the greater the FWHM of the fit curve, the less uniform the Fe³⁺-mSF alignment. Fe³⁺-mSF alignment uniformity significantly increased over time, shown by peak 202, with greatest uniformity (19°) observed after 24 h. No significant increase in directional uniformity was observed for non-Fe³⁺-chelated mSF in response to the externally applied 1.4 T magnetic field, suggesting the magneto-responsive properties are not innate to mSF but rather imparted by Fe³⁺ chelation. Successful Fe³⁺ chelation was confirmed via significantly increased detection of ferric iron in Fe³⁺-mSF X-ray photoelectroscopy (XPS) spectra as well as the appearance of an iron-oxygen bond in Fe³⁺-mSF Fourier-Transform Infrared spectroscopy (FTIR) relative to non-Fe³⁺-chelated mSF.

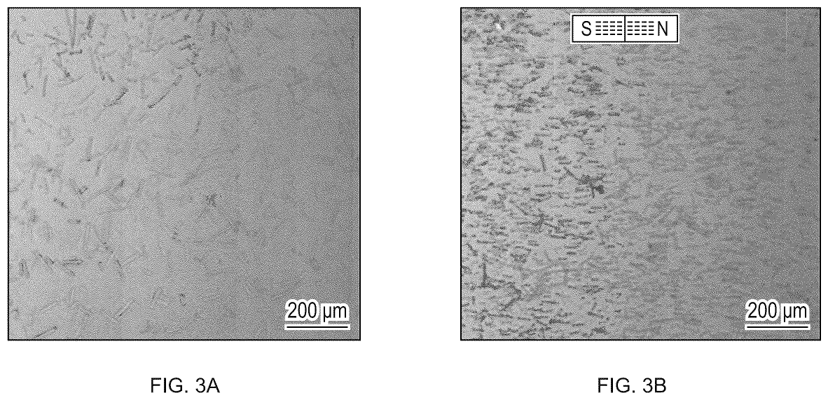

FIG. 3A shows non-ferric iron chelated mSF in absence of magnetic field, and FIG. 3B shows ferric iron-chelated mSF (‘Fe³⁺-mSF’) in 1.4 T magnetic field. For the magnetic response behavior of the microfibers to occur, the iron needs to be bound in some way to the silk microfibers. Chelating is an appropriate term to define the binding of iron to the mSF; and alternate mechanism may be employed to describe iron interacting with silk microfibers for imparting magnetic responsiveness.

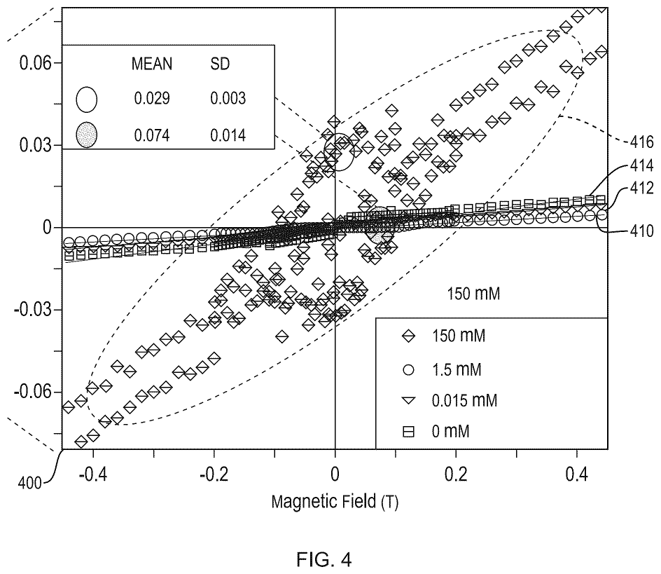

FIG. 4 shows a graph 400 of magnetic response of Fe³⁺-mSF when exposed to external magnetic fields. FIG. 4 graphs magnetization versus magnetic field curves for “no iron added” mSF (0.mM) 410, “low iron” (0.015 mM) 412, “moderate iron” (1.5 mM) 414, and “high iron” (150 mM) 416 Fe³⁺-mSF samples, with magnetic field ranging from −0.4 to 0.4 T at room temperature.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for forming a scaffold amenable to nerve cell regrowth, comprising:
providing a scaffolding compound based on a potential for regrowth of nerve tissue;
adding an iron solution to the scaffolding compound; and
washing the scaffolding compound to form a magneto responsive scaffold adapted for implantation adjacent damaged nerve tissue.

2. The method of claim 1 further comprising adding the magneto responsive scaffolding compound to a gel substance for allowing magnetically induced alignment, the alignment maintained by the gel substance.

3. The method of claim 1 further comprising binding iron from the iron solution to the scaffolding compound for imparting a magnetic response behavior to the scaffolding compound.

4. The method of claim 1 further comprising chelating iron from the iron solution to the scaffolding compound.

5. The method of claim 1 wherein the scaffolding compound is silk fibroin microfibers (mSF).

6. The method of claim 1 wherein the gel substance is a polymer.

7. The method of claim 4 wherein the gel substance is an injectable, in situ crosslinking hydrogel.

8. The method of claim 1 further comprising:
adding an aqueous solution of ferric chloride to the scaffolding compound;
agitating the scaffolding compound for 12-48 hours; and
washing with water for removing excess chloride compounds.

9. The method of claim 1 further comprising:
applying the microfiber medium in a therapeutic adjacency with an anatomical region for repair; and
applying a magnetic field to the microfiber medium for aligning the scaffold.

10. An injectable scaffold system for spinal cord injury treatment, comprising:
a silk fibroin microfiber (mSF) formed from a lyophilized powder and combined with an aqueous ferric chloride solution for imparting a magneto responsiveness to the mSF; and
an injectable crosslinking hydrogel combined with the magneto responsive mSF for introduction into an injury site.

11. The system of claim 10 further comprising a therapeutic magnetic source, the therapeutic magnetic source configured for applying between 100-400 mT for aligning the microfibers form a magnetic response, the crosslinking hydrogel adapted for maintaining the aligned orientation of the mSF following injection.

12. A method of forming an injectable medical scaffolding, comprising:
hydrolyzing silk fibroin in a sodium hydroxide solution to fabricate silk fibroin microfibers (mSF);
neutralizing the mSF with an acidic wash;
rinsing and lyophilizing the mSF to form a powder;
resuspending the mSF in a ferric chloride solution for 12-48 hours followed by water washing to form a magneto responsive mSF; and
combining the magneto responsive mSF with a hydrophilic crosslinking gel for maintaining magnetic alignment following introduction of a magnetic field.
